(19) United States
(12) Patent Application Publication
(10) Pub. No.: US 2008/0177362 A1
(43) Pub. Date: Jul. 24, 2008

(54) SCREENING DEVICE AND LEAD DELIVERY SYSTEM

(75) Inventors: William C. Phillips, Brooklyn Park, MN (US); John W. Forsberg, St. Paul, MN (US)

Correspondence Address: CAMPBELL NELSON WHIPPS, LLC, 408 ST. PETER STREET, SUITE 240, ST. PAUL, MN, 55102, US

(73) Assignee: MEDTRONIC, INC., Minneapolis, MN (US)

(21) Appl. No.: 11/693,328

(22) Filed: Mar. 29, 2007

Related U.S. Application Data
(60) Provisional application No. 60/885,599, filed on Jan. 18, 2007.

(51) Int. Cl. A61N 1/00

(52) U.S. Cl. 607/115

(57) ABSTRACT

Hand-held electrical signal generator screening device include a signal generator. The hand-held devices have incorporated stylets or are capable of receiving a stylet while a stylet is inserted into the lead. The devices allow for movement of the lead and verification of suitable placement of the lead via application of an electrical signal by one user.

### RELATED APPLICATION

This application claims the benefit of U.S. Provisional Application Ser. No. 60/885,599, filed Jan. 18, 2007, which application is hereby incorporated herein by reference in its entirety to the extent that it does not conflict with the disclosure herein.

### FIELD

This disclosure relates, inter alia, to implantable medical leads, medical electrical signal generators, stylets, steering devices, and methods of use thereof.

### BACKGROUND

Currently, implanting a medical lead in a patient for the purposes of neurostimulatory therapy requires multiple individuals and time consuming steps. The lead is typically implanted by a physician within the sterile field of an operating room. To verify that the lead is placed at a therapeutically effective location, the physician connects the lead, with a stylet still inserted, to a connector within the sterile field. The connector is coupled by a cable to an external neurostimulator located outside the sterile field. The neurostimulator is in telemetric communication with a programmer device that is also located outside the sterile field. A person, other than the implanting physician, operates the programmer outside the sterile field, instructing certain electrodes on the implanted lead to deliver electrical signal therapy to the patient. If, after a series of signals are applied to the patient via the various electrodes, the patient does not respond favorably, the physician removes the lead from the connector and moves the location of the lead within the patient. The physician then again connects the lead to the connector in the sterile field and the process is continued. This continues until the proper location of the lead is verified by a favorable patient response.

There is a need for improved systems and methods for implanting medical leads and verifying the leads are implanted in the proper location.

### BRIEF SUMMARY

The present disclosure describes, inter alia, hand-held signal generator devices that can be operated by an implanting physician within the sterile field to verify proper placement of a medical lead within a patient.

In an embodiment, a hand-held electrical signal generator screening device is described. The device includes a housing and electronics disposed in the housing. The electronics are capable of generating an electrical signal and controlling a parameter of the electrical signal. A control element is disposed on the housing. The control element is operably coupled to the electronics to provide input regarding the parameter of the electrical signal. The device further includes a contact operably coupled to the electronics. The housing further includes a connector region configured to engage at least a portion of a medical lead and at least a portion of a handle of a stylet while the stylet is inserted into the lead. At least a portion of the contact is positioned such that the contact electrically couples the electronics to the lead when the portion of the lead is engaged by the connector region.

In an embodiment, a hand-held electrical signal generator screening device is described. The device includes a housing and electronics disposed in the housing. The electronics are capable of generating an electrical signal and controlling a parameter of the electrical signal. A control element is disposed on the housing. The control element is operably coupled to the electronics to provide input regarding the parameter of the electrical signal. The device further includes a contact operably coupled to the electronics. The device includes a stylet having a distal end and a proximal end. The distal end protrudes from the housing and is configured to be inserted into a medical lead. The housing further includes a connector region configured to engage at least a portion of a medical lead while the stylet is inserted in the lead. At least a portion of the contact is positioned such that the contact electrically couples the signal generator to the lead when the portion of the lead is engaged by the connector region.

The present disclosure also describes, inter alia, methods for implanting a lead and for verifying the proper location of an implanted lead. The methods may be carried out by a single person within the sterile field of an operating environment.

In an embodiment, a method includes inserting a stylet coupled to a hand-held electrical signal generator screening device into a lumen of a lead; and securing a portion of the lead to the hand-held electrical signal generator screening device such that contacts of the screening device are electrically coupled to the contacts of the lead. The contacts of the lead are electrically coupled to electrodes of the lead. The method further includes steering the lead, with the assistance of the stylet, within a patient such that the lead electrodes are positioned at a target location in the patient; and activating a control element disposed on the screening device to cause the screening device to apply an electrical signal to the patient via one or more of the electrodes. The method may also include determining whether a condition of the patient has improved to a predetermined value as a result of the application of the electrical signal.

By providing devices, systems and methods that allow an individual user to both implant a lead and verify proper placement of the lead, overall time of surgery would decrease. Also, the dependence of having an additional person in the operating room will be reduced. These and other advantages will be readily understood from the following detailed descriptions when read in conjunction with the accompanying drawings.

### BRIEF DESCRIPTION OF THE DRAWINGS

FIG. 1 is a diagrammatic representation of a schematic of a side view of an exemplary device.

FIG. 2 is a diagrammatic representation of a schematic of a top view of an exemplary device.

FIG. 3A is a diagrammatic representation of a perspective view of a lead.

FIG. 3B is a diagrammatic representation of a schematic of a top view of a stylet.

FIG. 4 is a diagrammatic representation of a schematic of a top view of an exemplary device.

FIG. 5 is a diagrammatic representation of a schematic of a side view of an exemplary device.

FIG. 6 is a diagrammatic representation of a schematic of a top view of an exemplary device.

FIG. 7 is a diagrammatic representation of a schematic of a top view of an exemplary device.

FIG. 8 is a diagrammatic representation of a schematic of a top view of an exemplary device.

FIG. 9 is a diagrammatic representation of a schematic of a top view of an exemplary device.

FIG. 10 is a schematic representation of a longitudinal cross section of a device showing some components used in facilitating steering of a stylet.

FIG. 11 is a schematic representation of a top view of some components used in facilitating steering.

FIG. 12 is a schematic representation of a side view of a portion of a pull rod and stylet.

FIG. 13 is a diagrammatic representation of a schematic of a side view of an exemplary device.

FIG. 14 is a block diagram of some components or modules of electronics according to various embodiments.

FIG. 15 is a block diagram of some output components or modules according to various embodiments.

FIG. 16 is a block diagram of some input components or modules according to various embodiments.

FIG. 17 is a perspective view of a representation of an exemplary device.

FIG. 18 is a top view of the representation of the device shown in FIG. 17.

FIG. 19 is a perspective view of a representation of the connector region the device shown in FIG. 17.

FIG. 20 is an exploded view of the representation of the device shown in FIG. 17.

FIG. 21 is a perspective view of a representation of an exemplary device.

FIG. 22 is a flow diagram of an exemplary method of using an exemplary device.

The drawings are not necessarily to scale. Like numbers used in the figures refer to like components, steps and the like. However, it will be understood that the use of a number to refer to a component in a given figure is not intended to limit the component in another figure labeled with the same number.

### DETAILED DESCRIPTION

In the following detailed description, reference is made to the accompanying drawings that form a part hereof, and in which are shown by way of illustration several specific embodiments of devices, systems and methods. It is to be understood that other embodiments are contemplated and may be made without departing from the scope of spirit of the present invention. The following detailed description, therefore, is not to be taken in a limiting sense.

All scientific and technical terms used herein have meanings commonly used in the art unless otherwise specified. The definitions provided herein are to facilitate understanding of certain terms used frequently herein and are not meant to limit the scope of the present disclosure.

As used in this specification and the appended claims, the singular forms “a”, “an”, and “the” encompass embodiments having plural referents, unless the content clearly dictates otherwise. As used in this specification and the appended claims, the term “or” is generally employed in its sense including “and/or” unless the content clearly dictates otherwise.

As used herein, “signal generator” and “pulse generator” are interchangeable. It will be understood that a pulse generator may generate an electrical signal or a plurality of electrical signals that are not pulses.

“Physician”, as used herein, means a person certified to practice medicine. However, “physician” and “anyone performing or capable of performing a procedure or procedures to which a physician is referred to herein” will be considered equivalent for the purposes of the disclosure provided herein.

This disclosure relates, inter alia, to implantable medical leads, medical electrical signal generators, stylets, steering devices, and methods of use thereof. More particularly, hand-held electrical signal generator screening devices that may also be used to assist in placement of a lead within a patient are described. The devices may be used by a single individual, who can both place the lead and verify the proper placement of the lead, all within the sterile field of an operating environment. The present disclosure also describes methods for implanting a lead and for verifying the proper location of an implanted lead. The methods may be carried out by a single person within the sterile field of an operating environment.

1. Device

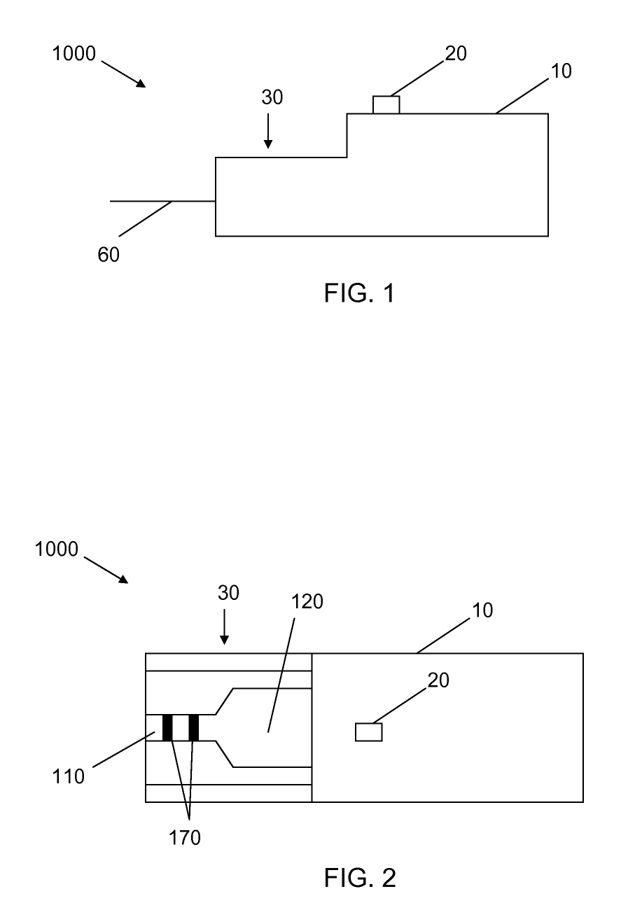

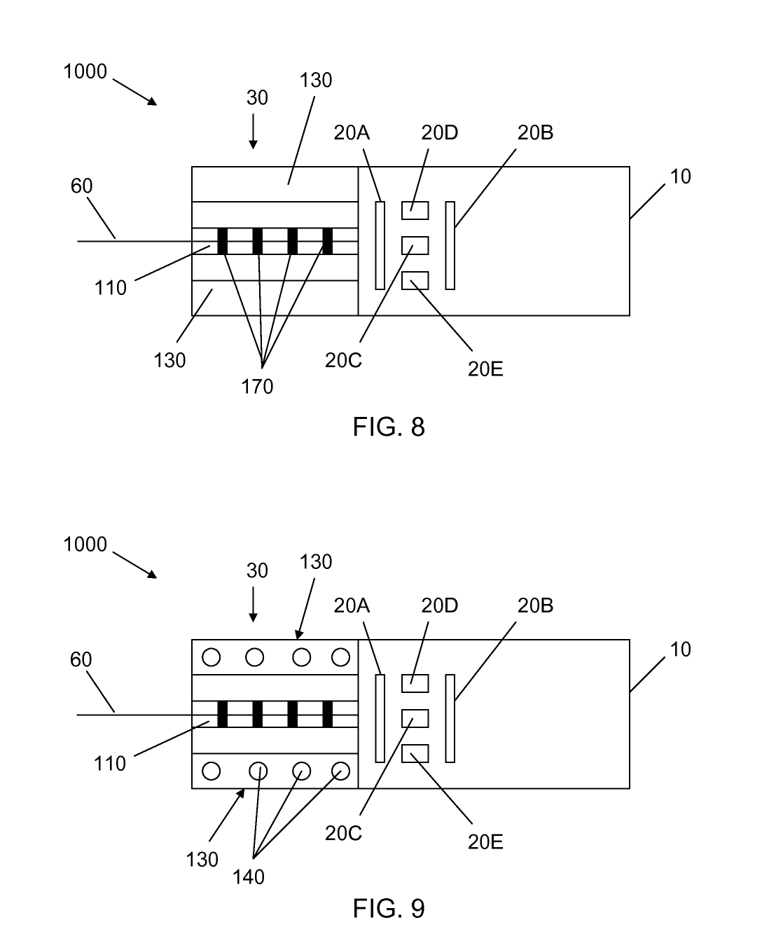

In FIGS. 1-11, diagrammatic representations of various exemplary devices 1000, systems or components thereof are shown. Device 1000 is a hand-held electrical signal generator screening device. Hand-held electrical signal generator screening device 1000 may be used to facilitate placement of any medical lead 40. For example, medical lead 40 may be used in conjunction with a cochlear implant; a pacemaker; a defibrillator; a neurostimulator, such as a spinal cord stimulator, a deep brain stimulator, a peripheral nerve stimulator, a vagal nerve stimulator, an occipital nerve stimulator, a subcutaneous stimulator, or the like; a gastric stimulator; or the like.

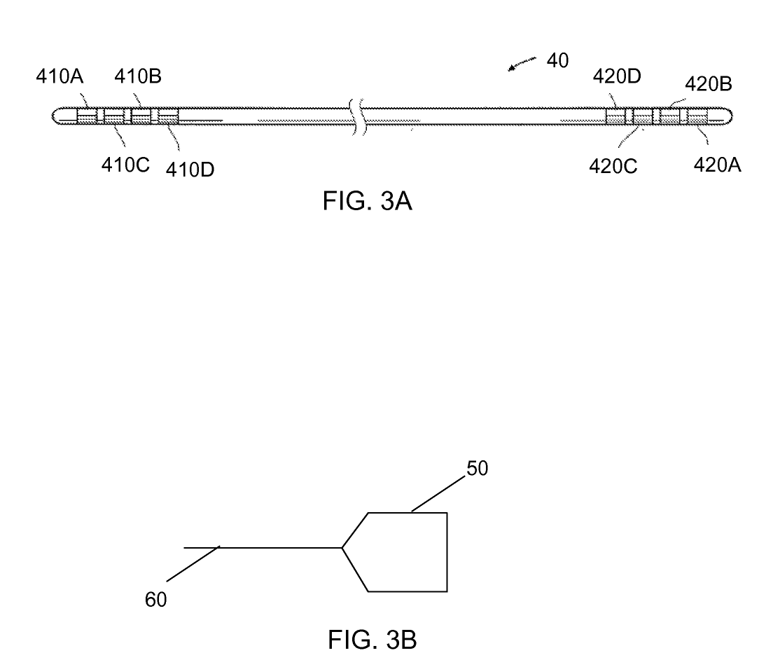

Device 1000 includes a housing 10 and electronics (not shown in FIGS. 1-11) disposed in housing 10. The electronics are capable of generating an electrical signal and controlling a parameter of the electrical signal. One or more control elements 20 are disposed on housing 10. Control element 20 is operably coupled to electronics to provide input regarding the parameter of the electrical signal. Device 1000 further includes one or more contacts 170 (see, e.g., FIGS. 3, 4, and 6-9) operably coupled to electronics. Housing 10 includes a connector region 30 configured to engage at least a portion of a medical lead 40 (see, e.g., FIG. 2A) while stylet 60 is inserted into lead 40. At least a portion of the one or more contacts 170 are positioned such that contact 170 electrically couples the electronics to lead 40 when the portion of lead 40 is engaged in the connector region 30. For example, contacts 170 may be positioned at or above a surface of connector region 30 that is configured to receive lead 40.

As shown in FIG. 3A, lead 40 includes one or more lead contacts 410 that are electrically coupled to one or more electrodes 420 via a conductor. Typically, the most proximal lead contact 410A is electrically coupled to the most distal electrode 420A, the most distal lead contact 410D is electrically coupled to the most proximal electrode 420D, etc. While lead 40 shown in FIG. 3A has four lead contacts 410A-D and four electrodes 420A-D, it will be understood that lead 40 may contain any number of contacts 410 or electrodes 420. It will be further understood that any type of lead 40; e.g. a surgical lead, a percutaneous lead, or the like, may be employed with the systems and devices described herein.

Referring to FIGS. 2, 4, and 6-9, which show top views of exemplary devices 1000, device 1000 may include a lead recess 110 configured to engage lead 40 while stylet 60 is disposed within lead 40. Lead 40 may be secured by lead recess 110 by press fit or any other suitable mechanism. Contacts 170 may be disposed in lead recess 110 such that contacts 170 electrically couple lead contacts 410 to electronics when lead 40 is engaged by lead recess 110. For example, at least a portion of each contact 170 may be positioned at or above the surface of lead recess 110 and may be longitudinally spaced apart in a substantially linear manner at a distance suitable for contacting lead contacts 410 when lead 40 is received by recess 110. Device 1000 may include any number of contacts 170. Preferably, device 1000 has at least as many contacts 170 as lead contacts 410 to which it is desired to provide an electrical signal. By way of example, device 1000 may include one, two, four, eight, or sixteen contacts 170. Device 1000 may include a lead locking mechanism (not shown) to further secure lead 40 in lead recess 110. Lead locking mechanism may releasably and lockingly engage connector region 30. By way of example, lead locking mechanism may be attached to connector region 30 via a hinge or may be a separate piece.

1a. Stylet and Steering

As shown in FIG. 3B, stylet 60 may include handle 50. Stylet 60 is configured to be inserted into lead 40 to facilitate placement and steering of lead 40, particularly electrodes 420, to a desired location in a patient. Stylet handle 50 may be grasped by an implanting physician and used to facilitate steering and placement. In various embodiments, stylet 60 and stylet handle 50 may be removably secured by device 1000. Thus, an implanting physician may choose to initially locate lead 40 in a patient using stylet 60 and stylet handle 50 and then engage stylet 60, stylet handle 50, and lead 40 in device 1000 to verify that lead is placed in a therapeutically effective location. If lead 40 location in the patient needs to be changed, the physician may remove stylet 60, stylet handle 50, and lead 40 from device 1000 and maneuver lead 40 within the patient. Alternatively, the physician may maneuver distal portion of lead 40 within the patient while lead 40, stylet 60, and stylet handle 60 are engaged in device 1000.

Referring to FIGS. 2, 6 and 7, device 1000 may include a handle recess 120 configured to receive at least a portion of a stylet handle 50 (see, e.g., FIG. 3A). Device 1000 shown in FIGS. 2, 6 and 7 is capable of receiving stylet handle 50 while stylet 60 is inserted into lead 40. Handle recess 120 may be shaped in a complementary manner to stylet handle 50; e.g., such that recess 120 snuggly receives at least a portion of handle 50. Device 1000 may further include a stylet locking mechanism (not shown) to further secure stylet handle 50 in handle recess 120. Stylet locking mechanism may releasably and lockingly engage connector region 30. By way of example, stylet locking mechanism may be attached to connector region 30 or other portion of housing 10 via a hinge or may be a separate piece. Stylet locking mechanism and lead locking mechanism may be a single piece.

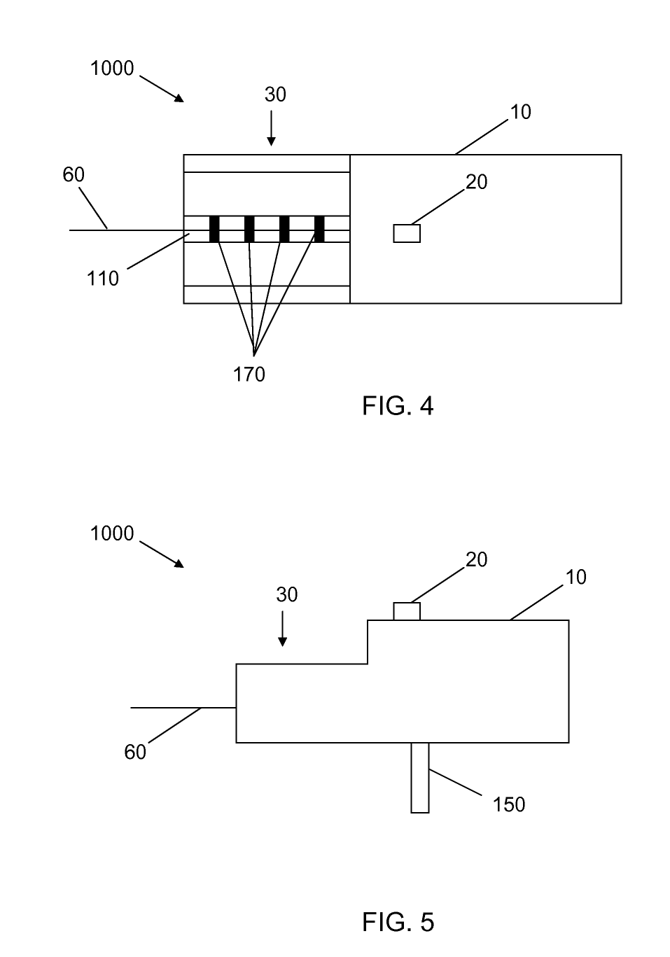

Referring to FIGS. 4, 8 and 9, stylet 60 may be connected to device 1000. By way of example, stylet 60 may be fastened, adhered, bonded, or otherwise connected to device 1000. In such embodiments, a stylet handle recess may be omitted from device 1000. Distal end of stylet 60 protrudes from housing 10 and is configured to be inserted into medical lead 40. Connector region 30 may include a lead recess 110 configured to engage lead 40 while stylet 60 is inserted into lead 40.

Referring to FIG. 5, device may include a steering mechanism actuator 150 operably coupled to stylet 60. Actuation or activation of steering mechanism actuator 150 is capable of causing bending of distal end portion of stylet 60, allowing stylet 60 to be used to steer lead 40 into position with a patient. Steering mechanisms and steering mechanism actuators or activators 150 may be employed in device 1000 embodiments capable or receiving stylet 60 and stylet handle 50 or in embodiments where stylet 60 is connected to device 1000. Steering mechanism actuator or activator 150 may be placed anywhere on device 1000. Preferably, steering mechanism actuator or activator 150 is placed in a location that allows for ergonomical activation or actuation. Steering mechanism actuator or activator 150 may take the form of any suitable acuatable mechanism, such as a push button, a dial, a wheel, a slide bar, joy stick, a switch, a trigger, or the like. Alternatively, steering mechanism actuator or activator 150 may take the form of a touch screen display or the like. In FIG. 5 steering mechanism actuator 150 is in the form of a trigger or slide bar. Any suitable mechanism for causing stylet 60 to bend and facilitate steering of lead 40 may be used. In various embodiments, the direction of the bending of stylet 60 upon activation of the steering mechanism may be controlled by the user. Alternatively, the direction of bending may be fixed, and device 1000 may be rotated to achieve steering in a particular direction.

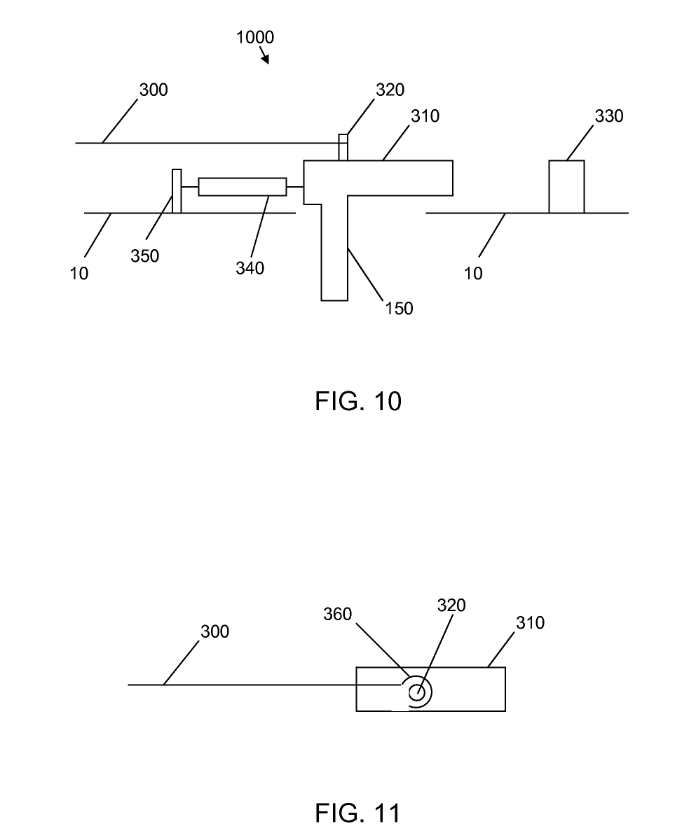

By way of example, and referring to FIGS. 10-12, a pull rod 300 may be connected to distal end portion of stylet 60 to cause stylet 60 to bend when pull rod is retracted. Examples of devices and systems having pull rods that may be readily employed in a device 1000, 2000 as described herein are described in U.S. Pat. No. 5,327,906, entitled STEERABLE STYLET HANDLE, and U.S. Pat. No. 6,338,725, entitled LARGE-DIAMETER INTRODUCER SHEATH HAVING HEMOSTASIS VALVE AND REMOVABLE STEERING MECHANISM, which patents are hereby incorporated herein by reference in their entirety to the extent that they do not conflict with the present disclosure.

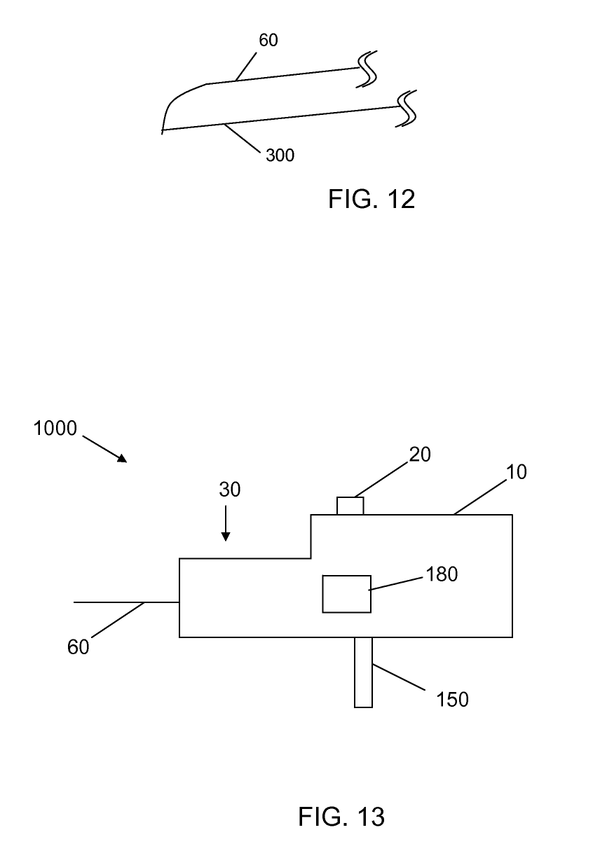

Referring to FIG. 10, a schematic representation of a longitudinal cross section of a portion of device 1000 in which steering mechanism actuator 150 is coupled to pull rod 300, which is coupled to distal portion of stylet 60 (see FIG. 12). Actuation of steering mechanism actuator 150 causes pull rod 300 to retract, bending the distal portion of stylet 60. The degree of bending may be controlled the degree of actuation of steering mechanism actuator 150. As shown in FIG. 10, pull rod 300 may be coupled to post 320 on surface 310 of actuator 150. Of course, pull rod 300 may be coupled to actuator 150 via any suitable means, such as a fastener, e.g. a screw, an adhesive or other bonding agent, or the like. A stop 330 may be formed in housing 10 or attached to housing 10 to prevent actuator 150 from moving distally beyond a predetermined distance. Spring 340 may be used to bias actuator 150 proximally in housing 10. Spring 340 may be coupled to housing 10; e.g. through a post 350 formed from housing 10 or attached to housing 10, and may be coupled to actuator 150. It will be appreciated that various additional components may be included in the steering mechanism depicted in FIG. 10 to facilitate operation of the mechanism.

FIG. 11 is a top view of actuator 150 and pull rod 300 shown in FIG. 10, showing surface 310 of actuator 310. Pull rod 300 includes a loop 360 or fastener for retaining pull rod 300 relative to post 320 on surface 310 of actuator 150. Loop 320 may be formed from pull rod 300 or may be attached to pull rod 300. Such a configuration allows for stylet 60 or pull rod 300 to be removed from device 1000, allowing for exchange of stylets 60 of different thicknesses, stiffnesses, or the like. Of course loop 360 and post 320, fastener or the like may be in any suitable form for pull rod 300 to be removable from device 1000. In various embodiments, post 320 is a fasterner, such as a screw. In embodiments where pull rod 300 is not removable, pull rod 300 may be attached to actuator 150 through any suitable mechanism.

Referring to FIG. 13, housing 10 of device 1000 may comprise an opening 180 configured to allow stylet 60 to be removed or inserted into device 1000. In various embodiments, stylet 60 may be removed or inserted through opening 180 while lead 40 is engaged in connector region 30 of device 1000. Accordingly, stylet 60 may be inserted into or withdrawn from opening 180 and into or from lead 40 in a single step.

1b. Control element

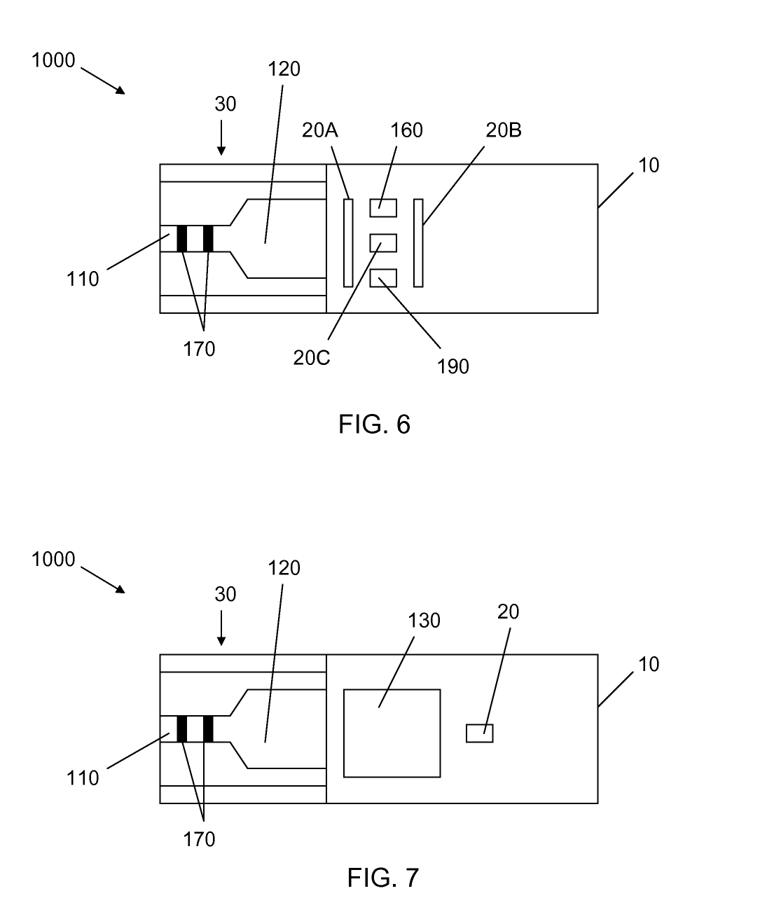

Hand-held electrical signal generator screening device 1000 may be used to control one or more parameter of an electrical signal generated by the electronics. Parameters include: which contacts of the device (and thus which electrodes of the lead) are receiving the signal; the amplitude, frequency, pulse width, or polarity of the signal; etc. Referring to FIGS. 6-9, one or more control elements 20, 20A, 20B, 20C, 20D, 20E are disposed on housing 10 of device 1000. Control element 20, 20A-E is operably coupled to electronics (not shown in FIGS. 6-9). Each of the control elements 20, 20A-E may be used to control a given parameter or a given set of parameters. Control element 20, 20A-E may comprise an actuatable mechanism, such as a push button, a dial, a wheel, a slide bar, joy stick, a switch, a trigger, or the like. Control element 20, 20A-E may comprise a touch screen display or the like.

Control element 20, 20A-E may allow a user to select one or more of a plurality of contacts 170 of device 1000 to receive an electrical signal. This may be done in a trolling manner, e.g. with a slide bar or wheel.

Control element 20, 20A-E may allow the user to control the amplitude of the signal being delivered through the use of a slide bar or scroll wheel, for example. In verifying that the lead is in a proper location, amplitude of the signal is typically started low and then ramped up until the patient indicates that a particular sensation threshold has been achieved.

In certain embodiments, it may be desirable to limit the number of control elements 20, 20A-E and parameters to be adjusted to simplify device 1000. For example if device 1000 is used to implant a lead 40 for spinal cord stimulation for treatment of pain, it may not be necessary for the user to control pulse width and frequency of the signal via the device.

It may be desirable to have a separate control element 20, 20A-E, such as a push button, to deliver the electrical signal as a safety measure. For example, the electrical signal may only be delivered when the button is pressed—when the button is released, the signal is not delivered.

1c. Impedance Check Element

Referring to FIG. 6, device 1000 may include an impedance check element 160 operably coupled to the electronics. Impedance check element 160 may take the form of any actuatable or activatable mechanism. Impedance check element 160 allows the user to check the integrity of lead 40 coupled to contact 170. An indicator may signal to the user whether the integrity of lead 40 with regard to a given electrode 420 is in working order.

A number of ways to check the integrity of lead 40 are envisioned. For example, actuation of an impedance check element 160, such as a button, could start a sequence for checking all major combinations of electrodes 420 to determine lead integrity. In some embodiments, the integrity of only active electrodes 420 (i.e., those currently selected) are checked. Alternatively, it is possible to manually select combinations using a mechanism, such as a slide, to select the electrode 420 combinations; e.g., 0-1, slide, 1-2, slide, 2-3, slide, etc.

1d. Input Element

Referring again to FIG. 6, device 1000 may include an input element 190 operably coupled to electronics. Input element 160 may take the form of any actuatable or activatable mechanism. Input element 190 may be disposed on housing 10. In an embodiment, input element 190 is used to relay information regarding a patient's response to an applied electrical signal to the electronics to optimize the parameters associated with the signal for treating a disease state of the patient.

1e. Indicator

Referring to FIGS. 7-9, device 1000 may include an indicator 130 disposed on housing 10. Indicator 130 provides an audio or visual indication to the user of device 1000 as to what is occurring with device 1000. For example, indicator 130 may indicate to which of the plurality of contacts 170 an electrical signal is being applied. Indicator 130 may comprise a plurality of indicator elements 140 (see, e.g. FIG. 9). The number of indicator elements 140 may be equal in number to the number of contacts 170. As shown in FIG. 9, the number of indicator elements 140 disposed on a given side of connector region 30 may be equal in number to the number of contacts 170. Each indicator element 140 may correspond to one of the plurality of contacts 170 and is capable of indicating whether an electrical signal is being applied to the corresponding contact 170. The indicator elements 140 may be spaced apart in a manner substantially similar to the spacing of contacts 170. Such an arrangement may be desirable to mimic the arrangement of the corresponding contacts 170 of device 1000. In various embodiments, contacts 170 are longitudinally spaced apart in a substantially linear manner, and indicator elements 140 are longitudinally spaced apart in a substantially linear manner in the same direction as contacts 170. However, it may be desirable for indicator elements 140 to correspond to the electrodes 420 of lead 40 to which an electrical signal is being applied. As lead 40 may have the most proximal contact 410A electrically coupled to the most distal electrode 420A, the most distal contact 410D electrically coupled to the most proximal electrode 420D, etc. (see, e.g. FIG. 4), it may be desirable for indicator elements 140 to be longitudinally spaced apart in a substantially linear manner in the opposite direction of the device contacts 170. As such, indicator elements 140 may correspond to lead electrodes 420 in the same direction (proximal to distal) as lead electrodes 420.

Indicator elements 140 may be any suitable indicator element allowing a user to determine which contacts 170 or electrodes 420 are receiving an electrical signal. In an embodiment, indicator element 140 comprises a light emitting element, such as a light emitting diode. Light emitting element, or other suitable indicator element 140, may be configured to emit more intense light as the amplitude of the electrical signal being applied to the corresponding contact 170 or electrode 420 is increased; to emit light of a first color if the corresponding contact 170 or electrode 420 is receiving an electrical signal having a positive polarity; to emit light of a second color if the corresponding contact 170 or electrode 420 is receiving an electrical signal having a negative polarity; to emit a flashing light at a rate indicative of the frequency of the stimulation signal; etc.; or the like.

Indicator 130 may be operably coupled to contact 170, to electronics, or to any component of device 1000 that is capable of relaying information to indicator 130 regarding how device 1000 is performing.

In various embodiments, indicator 130 is capable of indicating which segments of a radially segmented electrode are receiving an electrical signal. For example, indicator elements 140 may be radially disposed in, on, or about housing 10.

Indicator 130 may comprise a display, such as an LCD display (see, e.g., FIG. 7)

1f. Electronics

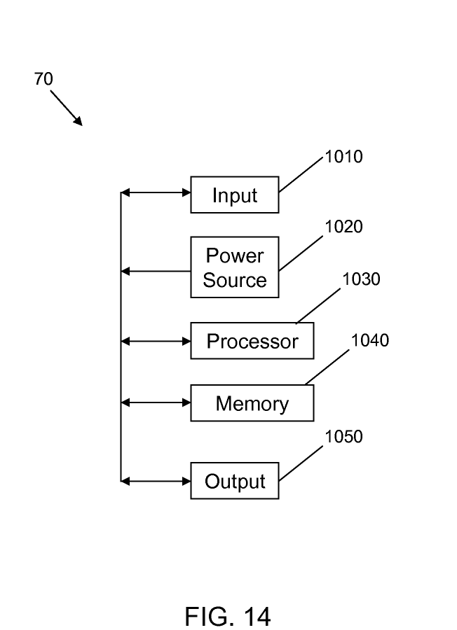

Device 1000 includes electronics capable of generating an electrical signal, in response to one or more control element 20, to contact 170, and thus to lead contact 410 and lead electrode 420 to a patient. Referring to FIG. 14, a block diagram of an embodiment of electronics 70 of a device 1000 is shown. Electronics 70 includes an input 1010, output 1050, power source 1020, and processor 1030. Electronics may also include a memory 1040. Processor 1030 may be programmed to receive information from input 1010 to modify one or more parameters of electrical stimulation signal, such as frequency, amplitude, or pulse width. Algorithms for modifying electrical stimulation signal in response to information from input 1010 may be stored in processor 1030 or memory 1040. Processor 1030 may direct information from input 1010 to be stored in memory 1040. All components may be configured on one or more Application Specific Integrated Circuits (ASICs), a combination of ASICs and commercially available integrated circuits, or the like, except the power source 1020. All components may be connected to a bi-directional data bus that is non-multiplexed with separate data lines, except the power source 1020.

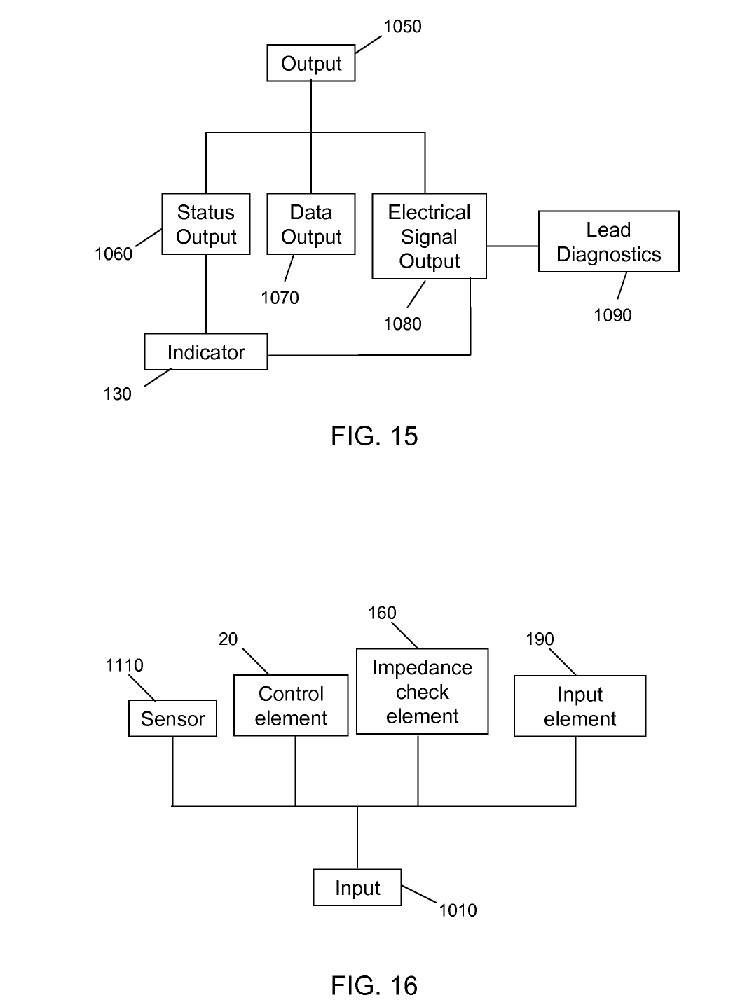

One or more input 1010 or output 1050 modules may be included in electronics 70. FIG. 15 shows various output 1050 modules, which may be the same or different modules. The output 1050 modules may include status output 1060, data output 1070, and electrical signal output 1080. Electrical signal output 1080 may be operably coupled to a lead diagnostics 1090 module, e.g. for testing impedance, or to indicator 130, e.g. for providing information regarding the electrical signal. Status output 1060 module may be operably coupled to indicator 130. Status output 1060 module, data output 1070 module, or electrical signal output 1080 module may be operably coupled, either wirelessly or via wire, to a second device, such as a display, computer, or the like. While not shown, it will be understood that data output 1070 module may be operably coupled to indicator 130. FIG. 16 shows various input 1010 modules that may be the same or different. Input 1010 modules may include one or more sensors 1110; control elements 20, impedance check elements 160, or input elements 190.

Power source 1020 may include a rechargeable cell, a primary cell, or coin cells, such as AA, AAA, or AAAA batteries. Power source 1020 is rechargeable, the power cell may be recharged inductively, by contact to a base station, may contain a removable battery pack, or the like. If it is desirable to keep the unit hermetic, e.g. for sterilizing, it may be desirable to have an inductively rechargeable cell. Power source 1020 may include a cable to plug into an outlet. It may be desirable for the cable to be of medical grade for patient safety.

Memory 1040 is capable of collecting data, via processor 1030, data bus, etc., associated with device 1000 or implant procedure. The collection of data during an implant procedure may be used post-operatively to facilitate programming of an implantable signal generator to be implanted in the patient. The collected data may be used by a physician to aid in more accurate placement of leads and configuration in future implants. By being in control of electrode settings and placement in the operating room, a physician can learn over time what lead placement is most beneficial for a given type of indication to be treated.

Input 1010 or output 1050 modules may include a wireless module or wired port. Exemplary wireless modules include bluetooth, telemetry, or the like.

1g. Form factor

Device 1000 or components of device 1000 may be disposable. It may be desirable for device 1000, or components thereof, to be disposable if device 1000 is to be used in a sterile field of an operating environment. Alternatively, device 1000, or components thereof, may be sterilizable or resterilizable, allowing device 1000 to be used multiple times in a sterile field. It may be desirable for electronics 70, or components thereof, to be removable to aid in the sterilization or resterilization of device 1000.

If device 1000, or components thereof, are disposable, it may be desirable to make housing 10 or components thereof of a plastic material. If device 1000 or components are to be sterilizable, it may be desirable to make housing 10 or other device 1000 components of a metallic material.

Contacts 170 of device 1000 are made of a conductive material. For example, contact 170 may be AuNi plated.

Device 1000 may be configured to receive any number of leads 40, such as one, two, three or more leads.

In various embodiments, device 1000 may be remotely controlled by a person other than the implanting physician. Wired or wireless connectivity may be employed to achieve such a result.

1h. Exemplary embodiments

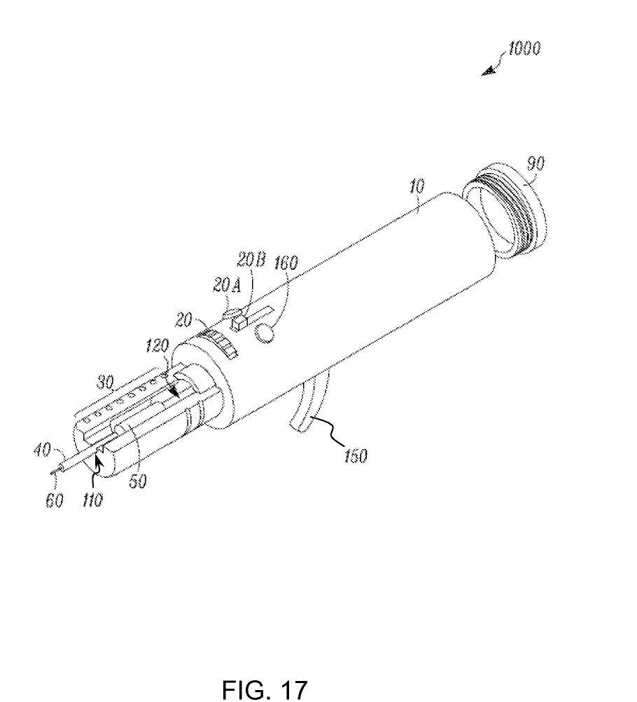

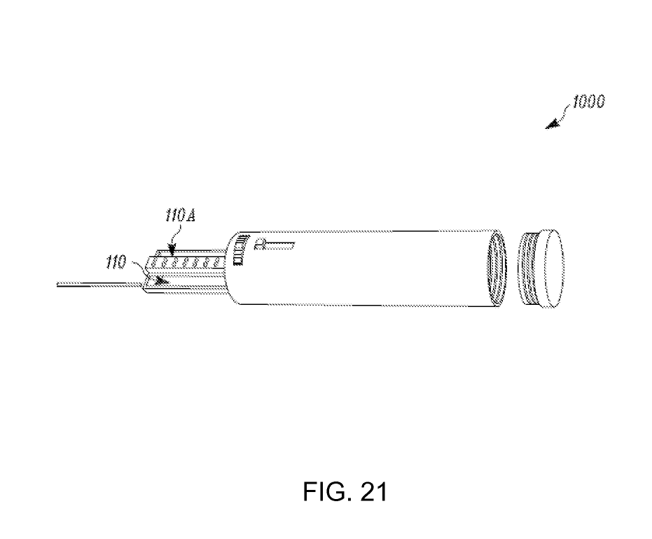

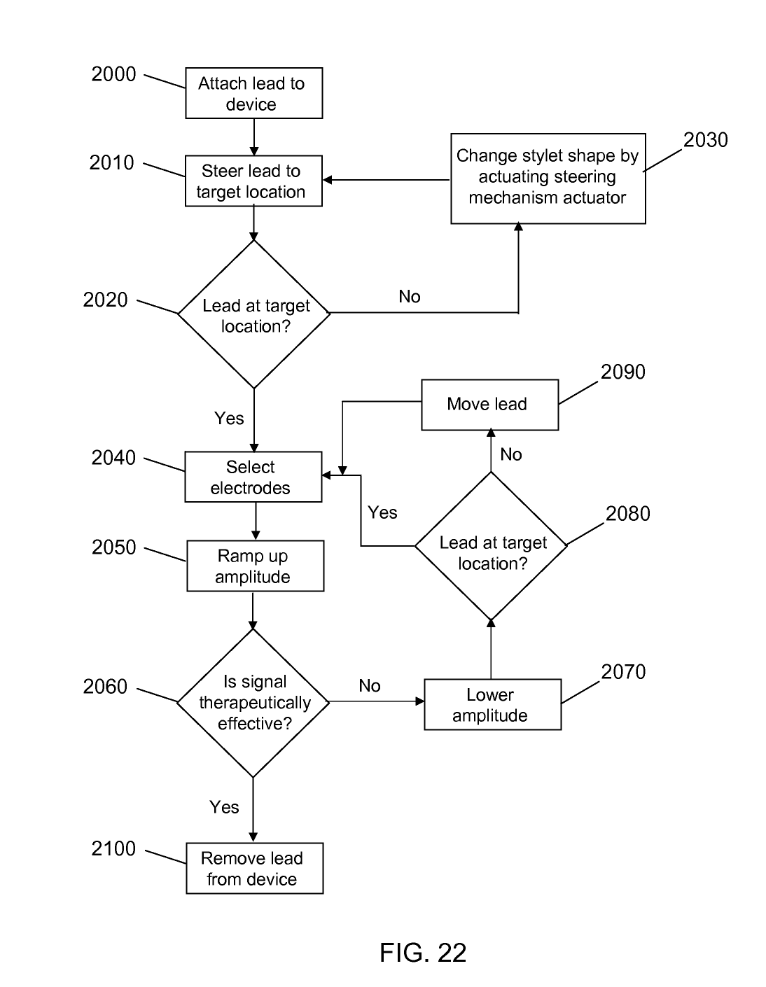

Referring to FIGS. 17-21, representative embodiments of devices are shown. FIGS. 17-20 illustrate a first representative embodiment, and FIG. 21 illustrates a second representative embodiment.

Referring to FIG. 17, a representative example of a device 1000 configured to receive one lead 40 is shown. Device 1000 comprises housing 10, which comprises connector region 30. Connector region 30 comprises lead recess 110 configured to receive and engage lead 40. Connector region 30 further comprises stylet handle recess 120 configured to receive and engage handle 50 of stylet 60. Control elements 20, 20A, 20B are disposed on housing 10, as are steering mechanism actuator 150 and impedence check element 160.

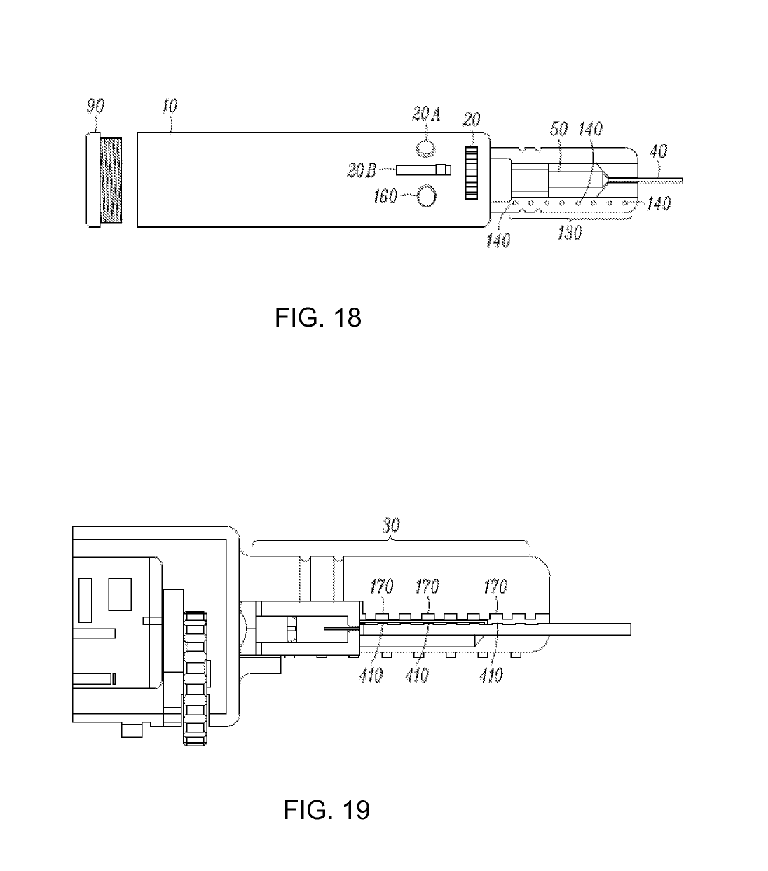

FIG. 18 provides a view top view of device 1000 as depicted in FIG. 17. In the top view, indicator 130 and indicator elements 140, which are longitudinally spaced apart LED elements are identified. The LED elements are arranged in orientation with electrodes 420 of lead 40, such that the most proximal LED element corresponds with the most proximal electrode 420 on lead 40. Control element 20B is a slide that allows a user to select which electrodes 420 are to receive a stimulation signal by trolling. When slide 20B is in the most proximal position, the most proximal lead electrode 420 is selected to receive an electrical signal, when slide 20B is in the most distal position; the most distal lead electrode 420 is selected to receive an electrical signal; etc. Control element 20 is a scroll wheel that allows a user to increase or decrease the amplitude of the electrical signal applied. Control element 20A is a push button that allows a user to control whether an electrical signal is applied. If push button 20A is depressed, a signal may be applied. If push button 20A is not depressed a signal may not be applied.

End cap 90 is securable and removable from housing 10 and provides electronics access to housing 10.

FIG. 19 is a close-up perspective view of connector region 30 of device shown in FIG. 17, where contacts 170 of device 1000 are shown in alignment with lead contacts 410.

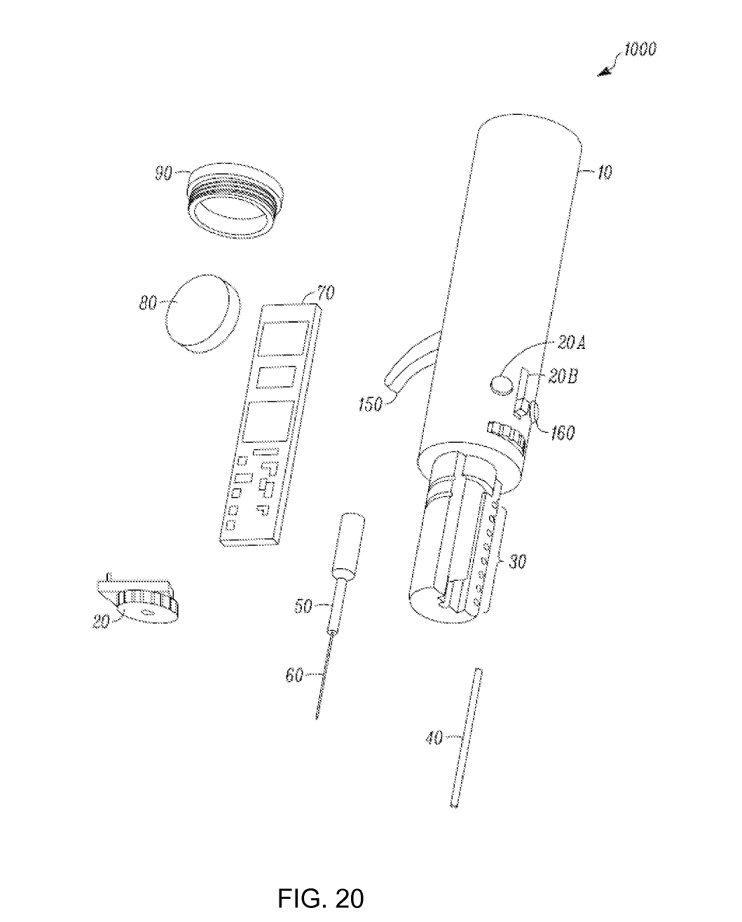

FIG. 20 is an exploded view of the device 1000 shown in FIG. 19. In FIG. 20, electronics 70, including power cell 80 serving as power source, which are contained within housing 10 during use, are shown.

Referring to FIG. 21, a representative device 1000 configured to receive two leads is shown. Device 1000 comprises two lead recesses 110, 110A.

2. Method

In an embodiment, a method includes inserting a stylet coupled to a hand-held electrical signal generator screening device into a lumen of a lead; and securing a portion of the lead to the hand-held electrical signal generator screening device such that contacts of the screening device are electrically coupled to contacts of the lead. Contacts of the lead are electrically coupled to electrodes of the lead. The method further includes steering the lead, with the assistance of the stylet, within a patient such that the lead electrodes are positioned at a target location in the patient; and activating a control element disposed on the screening device to cause the screening device to apply an electrical signal to the patient via one or more of the electrodes. The method may also include determining whether a condition of the patient has improved to a predetermined value as a result of the application of the electrical signal.

Referring to FIG. 22, a representative embodiment of a method that may be carrier our by an individual person is shown. By way of example, the method depicted in FIG. 22 will be discussed in conjunction with device 1000 as depicted in FIG. 17. The method includes attaching a lead 40 to device 1000 (2000), which may be accomplished by inserting stylet 60 into lead 40 and securing lead 40 in lead recess 110. The lead 40 is then attempted to be steered to a target location in a patient (2010) and a physician makes a determination of whether the lead is properly placed at the target location (2020). If the physician believes that the lead is not properly placed, the physician may change the shape of stylet 60 by actuating steering mechanism actuator 150 (2030) and again attempting to steer lead to the target location (2010). If the physician believes the lead 40 is properly placed, the physician may select electrodes 420 to which an electrical signal is to be applied (2040). This may be accomplished by moving slide 20B. The amplitude of the signal may then be increased to a predetermined maximum or until a patient responds favorably to the applied signal (2050). The amplitude may be increased by turning scroll wheel 20. The physician then determines whether the applied signal is therapeutically effective (2060); e.g., by feedback from the patient. If the signal is not therapeutically effective, the amplitude is lowered (2070); e.g., by moving scroll wheel 20B. The physician may then again determine whether the lead 40 is at the target location (2080). If the lead 40 is at the target location, different electrode(s) 40 may be selected (2040) and the amplitude ramped up (2050). A determination as to whether the signal is therapeutically effective (2060) may again be determined. If the lead 40 is determined not to be at the target location (2080), the lead 40 may be moved and steps 2040-2060 may be repeated. If the signal is determined to be therapeutically effective (2060), the lead 40 may be removed from the device 1000 (2100). the lead 40 may be removed from the device 1000 such that the stylet 60 remains with the device.

Thus, embodiments of the SCREENING DEVICE AND LEAD DELIVERY SYSTEM are disclosed. One skilled in the art will appreciate that the present invention can be practiced with embodiments other than those disclosed. The disclosed embodiments are presented for purposes of illustration and not limitation, and the present invention is limited only by the claims that follow.

What is claimed is:

1. A handheld electrical signal generator screening device comprising:
a housing,
electronics disposed in the housing, the electronics capable of generating an electrical signal and controlling a parameter of the electrical signal;
a control element disposed on the housing and being operably coupled to the electronics to provide input regarding the parameter of the electrical signal; and
a contact operably coupled to the electronics;
wherein the housing comprises a connector region configured to engage at least a portion of a medical lead and at least a portion of a handle of a stylet while the stylet is inserted into the lead, and
wherein at least a portion of the contact is positioned such that the contact electrically couples the electronics to the lead when the portion of the lead is engaged by the connector region.

2. The device of claim 1, wherein the connector region comprises a lead recess configured to receive the portion of the lead and wherein the portion of the contact is located in the lead recess.

3. The device of claim 1, wherein the connector comprises a handle recess configured to receive the portion of the stylet handle.

4. The device of claim 1, wherein the device comprises a plurality of contacts, wherein at least a portion of each of the plurality of contacts such that each contact is capable of electrically coupling the electronics to the lead when the portion of the lead is engaged by the connector region.

5. The device of claim 4, further comprising an indicator disposed on the housing, the indicator capable of indicating to which of the plurality of contacts an electrical signal is being applied.

6. The device of claim 5, wherein the plurality of contacts are spaced apart and wherein the indicator comprises a plurality of indicator elements, wherein each indicator element corresponds to one of the plurality of contacts and wherein each indicator element is configured to indicate whether an electrical signal is being applied to the corresponding contact.

7. The device of claim 6, wherein the plurality of indicator elements are spaced apart in a manner substantially similar to the spacing of the contacts.

8. The device of claim 6, wherein the contacts are longitudinally spaced apart in a substantially linear manner and wherein the plurality of indicator elements are longitudinally spaced apart in a substantially linear manner in substantially the opposite direction as the corresponding contacts.

9. The device of claim 6, wherein the indicator elements comprise light emitting elements.

10. The device of claim 9, wherein the light emitting elements are configured to emit more intense light as the amplitude of the electrical signal being applied to the corresponding contact is increased.

11. The device of claim 9, wherein the light emitting elements are configured to emit light of a first color if the corresponding contact is receiving an electrical signal having a positive polarity and to emit light of a second color if the corresponding contact is receiving an electrical signal having a negative polarity.

12. The device of claim 5, wherein the indicator is operably coupled to the contact.

13. The device of claim 5, wherein the indicator is operably coupled to the electronics.

14. The device of claim 1, wherein the control element comprises an actuatable mechanism.

15. The device of claim 1, further comprising an input element operably coupled to the electronics.

16. The device of claim 15, wherein the electronics are capable of receiving input information from the input element regarding patient response to the stimulation signal and using the information to optimize parameters associated with the signal for treating a disease state of the patient.

17. The device of claim 4, wherein the control element is configured to allow a user to select one or more of the plurality of contacts to receive an electrical signal.

18. The device of claim 1, wherein the control element is configured to allow a user to vary amplitude, polarity, pulse width, or frequency of the electrical signal.

19. The device of claim 1, further comprising an impedance check element operably coupled to the electronics, wherein the impedance check element allows the user to check the impedance of the lead coupled to the contact.

20. The device of claim 1, wherein the electronics are capable of controlling a plurality of parameters of the electrical signal and wherein the device comprises a plurality of control elements each configured to allow a user to control one or more of the plurality of parameters of the electrical generator.

21. The device of claim 1, wherein the electronics comprise a memory capable of storing input from the lead, device, or a user of the device.

22. The device of claim 1, further comprising a data output module configured to transfer stored information from the memory of the handheld device to a second device.

23. A handheld electrical signal generator screening device comprising:
a housing,
a signal generator contained within the housing;
a control unit contained within the housing and operably coupled to the signal generator, the control unit capable of controlling a parameter of an electrical signal generated by the signal generator;
a first control element disposed on the housing and being operably coupled to the control unit to provide input to the control unit regarding the parameter of the electrical signal;
a contact operably coupled to the signal generator;
a stylet having a distal end and a proximal end, the distal end protruding from the housing and configured to be inserted into a medical lead;
wherein the housing comprises a connector region configured to engage at least a portion of a medical lead while the stylet is inserted into the lead, and
wherein at least a portion of the contact is positioned such that the contact electrically couples the signal generator to the lead when the portion of the lead is engaged in the connector region.

24. The device of claim 23, further comprising an acutatable steering mechanism operably coupled to the stylet and capable of causing bending at least a portion of the distal end of the stylet when the mechanism is actuated.

25. A method comprising:
inserting a stylet coupled to a hand-held electrical signal generator screening device into a lumen of a lead;
securing a portion of the lead to the hand-held electrical signal generator screening device such that contacts of the screening device are electrically coupled to the contacts of the lead, the contacts of the lead being electrically coupled to electrodes of the lead;
steering the lead, with the assistance of the stylet, within a patient such that the lead electrodes are positioned at a target location in the patient;
activating a control element disposed on the screening device to cause the screening device to apply an electrical signal to the patient via one or more of the electrodes;
wherein the method is carried out by a single user within a sterile field during.

26. The method of claim 25, further comprising determining whether a condition of the patient has improved to a predetermined value as a result of the application of the electrical signal.
